(19) United States
(12) Patent Application Publication
(10) Pub. No.: US 2017/0014221 A1
(43) Pub. Date: Jan. 19, 2017

(54) Bridging Stent Graft with Interlocking Features and Methods for Use

(71) Applicant: Sanford Health, Sioux Falls, SD (US)

(72) Inventor: Patrick W. Kelly, Sioux Falls, SD (US)

(21) Appl. No.: 15/214,181

(22) Filed: Jul. 19, 2016

Related U.S. Application Data
(60) Provisional application No. 62/194,262, filed on Jul. 19, 2015.

(51) Int. Cl. A61F 2/07; A61F 2/852; A61F 2/95; A61F 2/844; A61F 2/89

(52) U.S. Cl. CPC A61F 2/07; A61F 2/844; A61F 2/89; A61F 2/95; A61F 2/852; A61F 2002/072; A61F 2002/8483

(57) ABSTRACT

The present disclosure provides a stent graft comprising (a) a self-expandable stent structure and a graft covering positioned over the self-expandable stent structure, the self-expandable stent structure having a first end and a second end, wherein the self-expandable stent structure defines a lumen, and (b) at least one annular channel defined by at least one of the self-expandable stent structure and the graft covering and extending radially outward from the self-expandable stent structure or at least one annular protrusion defined by at least one of the self-expandable stent structure and the graft covering and extending radially inward from the self-expandable stent structure.

### RELATED APPLICATIONS

This application claims priority to U.S. Provisional Application No. 62/194,262 entitled “Bridging Stent Graft with Interlocking Features and Methods for Use,” filed on Jul. 19, 2015, which is hereby incorporated by reference in its entirety.

### BACKGROUND THE INVENTION

Aneurysms are characterized as a bulging in an artery that results in a thinning of the arterial wall that can lead to rupture. An aneurysm rupture is a potentially life threatening condition. To repair an aneurysm, the surgeon would traditionally remove the diseased arterial tissue and replace it with a cloth replacement tube. This approach is extremely invasive and not an option for some patients.

Endovascular techniques make use of catheters to deliver a stent graft to the diseased site by gaining arterial access through small incisions in the groin or arm. The stent graft bridges the aneurysmal segment of the artery by firmly anchoring in two adjacent healthy segments of arterial tissue. The stent graft is held open by a metal scaffold or “stent” and uses a cloth cover to form a conduit for blood flow that keeps the blood pressure from reaching the diseased tissue. Traditionally, this device has worked well for aneurysms that are in the straight segment of the descending thoracic aorta or in the infrarenal aorta. But stent grafts have been less effective in areas of the branches of the aortic arch, branches of the descending thoracic aorta, or near the iliac branch. In order to repair these branched areas, bridging stents may be utilized. Bridging stents are relatively small diameter stent grafts that span from the main body stent graft to the native branch vessel. These bridging stents have unique requirements from covered stents used for other purposes.

The length between the target branch vessel and the main body stent graft is unpredictable because of the difference in anatomy between patients and difficulty of performing accurate measurements with currently available imaging modalities. Known techniques to deal with this challenge include using two or more bridging stents overlapping in a target branch vessel. This permits the overall length of the resulting combined stent graft structure to be manipulated by varying the overlap length between the two bridging stents.

A common problem for the branch vessels of aortic aneurysms is stenosis. There are many reasons for stenosis formatoin, including an abrupt compliance transition. An abrupt compliance transition is created when a stent, which is stiffer than the artery in which the stent is deployed and oversized by 10-20% relative to the artery. The stretch created in the arterial tissue is pulsatile causing repetitive micro-tearing and inflammatory response. This inflammation can lead to a stenosis just distal to the bridging stent.

Stent grafts currently known in the art are designed for use in a single diseased vessel and not a branched aortic aneurysm. When a self-expanding covered stent is used in a single vessel, the vessel wall provides support to the stent graft along its length, whereas branched aortic aneurysms are large empty sacs which do not provide support to a bridging stent graft. Because of this, currently available covered stent grafts typically do not have adequate proximal or distal fixation to avoid catastrophic failure. Finally, the amount of outward radial force required to achieve adequate distal fixation in the target vessel often may lead to intimal hyperplasia just distal to the bridging stent graft. Such inadequacies in bridging stent design can lead to mortality and serious complications when repairing complex branched aneurysms. To improve the technique of overlapping bridging stent grafts, devices and methods utilizing interlocking features between the two overlapping bridging stent grafts are advantageous.

### SUMMARY OF THE INVENTION

The bridging stent graft disclosed herein may be used to exclude an aneurysm. For example, the bridging stent graft may be deployed such that a proximal end of the bridging stent graft may deployed in a previously-placed main body stent graft and such that a distal end of the bridging stent graft may be deployed in a native branch vessel.

Thus, in a first aspect, the present invention provides a stent graft comprising (a) a self-expandable stent structure and a graft covering positioned over the self-expandable stent structure, the self-expandable stent structure having a first end and a second end, wherein the self-expandable stent structure defines a lumen, and (b) at least one annular channel defined by at least one of the self-expandable stent structure and the graft covering and extending radially outward from the self-expandable stent structure or at least one annular protrusion defined by at least one of the self-expandable stent structure and the graft covering and extending radially inward from the self-expandable stent structure.

In a second aspect, the present invention provides a method for placement of a stent graft that includes: (a) introducing a guidewire into an arterial configuration via arterial access, (b) loading a delivery catheter containing the stent graft of the first aspect onto the guidewire, (c) moving the delivery catheter along the guidewire and introducing the delivery catheter into the arterial configuration via arterial access, and (d) deploying the stent graft into at least one of the arterial configuration and a lumen of a previously-placed stent graft.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 is a side perspective view of a stent graft, according to an example embodiment.

FIG. 2 is a detail cross-sectional view of one half of the second end of the stent graft, according to the example embodiment of FIG. 1.

FIG. 3 a side perspective view a stent graft with a plurality of barbs on the second end of the stent graft, according to an example embodiment.

FIG. 4 is a side perspective view of a stent graft with a plurality of annular channels, according to an example embodiment.

FIG. 5 is a side perspective view of a stent graft with a plurality of annular protrusions, according to an example embodiment.

FIG. 6 is a side perspective view of a stent graft with a plurality of annular channels defined by a plurality of sinusoidal stents, according to an example embodiment.

FIG. 7 is a front view of the stent graft taken along line A-A of FIG. 6, according to the example embodiment of FIG. 6.

FIG. 8 is a side perspective view of a stent graft with helical channels, according to an example embodiment.

### DETAILED DESCRIPTION OF THE INVENTION

Exemplary devices and methods are described herein. It should be understood that the word “exemplary” is used herein to mean “serving as an example, instance, or illustration.” Any embodiment or feature described herein as “exemplary” is not necessarily to be construed as preferred or advantageous over other embodiments or features. The exemplary embodiments described herein are not meant to be limiting. It will be readily understood that certain aspects of the disclosed systems and methods can be arranged and combined in a wide variety of different configurations, all of which are contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an exemplary embodiment may include elements that are not illustrated in the Figures.

As used herein, with respect to measurements, “about” means +/−5%.

As used herein, diameter ranges pertain to an unconstrained, ex vivo state of the stent graft and stent graft extensions. When the stent graft and stent graft extensions are in a deployed, in vivo state the diameter ranges will be on the order of about 10-20% smaller in diameter than the ex vivo state.

As used herein, “first end” refers to the end of the main body stent graft that will be a “proximal end” upon deployment in vivo through which blood flow enters the lumen of the stent graft.

As used herein, “second end” refers to the end of the main body stent graft that will be a “distal end” upon deployment in vivo through which blood flow exits the lumen of the stent graft.

As used herein, “passive fixation” refers to friction, interaction between the cloth of the grafts, radial strength of the stent structure and blood pressure that holds the component stent grafts together at the site of overlap.

As used herein, “active fixation” refers to features coupled to a stent, graft, or stent graft that may actively engage vasculature or another stent graft, including hooks, bi-directional hooks, stent structure elements, anchors, staples, bio-activated adhesive, or a combination thereof, among other possibilities.

As used herein, a “stent graft” is a tubular, radially-expandable device comprising a fabric supported by a stent, and may be used to bridge aneurysmal arteries. As such, the term stent graft may be used herein to include bridging stent grafts. Such stent grafts and methods for their deployment and use are known to those of skill in the art. For example, vascular sheaths can be introduced into the patient's arteries, through which items, including but not limited to, guidewires, catheters and, eventually, the stent graft, may be passed.

As used herein, a “stent” is typically a cylindrical frame and means any device or structure that adds rigidity, expansion force, or support to a prosthesis, while “stent graft” refers to a prosthesis comprising a stent and a graft material associated therewith that forms a lumen through at least a portion of the length of the stent. A “graft” is a cylindrical liner that may be disposed on the stent's interior, exterior or both. A wide variety of attachment mechanisms are available to join the stent and graft together, including but not limited to, sutures, adhesive bonding, heat welding, and ultrasonic welding.

The stent can be made of any suitable material, including but not limited to biocompatible metals, implantable quality stainless steel wires, nickel and titanium alloys, and biocompatible plastics. The stents can either have material properties necessary to exhibit either self-expanding or balloon-expanding characteristics.

Any suitable graft material can be used. In a preferred embodiment, the graft material is a biocompatible fabric, including but not limited to woven or knitted polyester, such as poly(ethylene terephthalate), polylactide, polyglycolide and copolymers thereof; fluorinated polymers, such as PTFE, expanded PTFE and poly(vinylidene fluoride); polysiloxanes, including polydimethyl siloxane; and polyurethanes, including polyetherurethanes, polyurethane ureas, polyetherurethane ureas, polyurethanes containing carbonate linkages and polyurethanes containing siloxane segments. Materials that are not inherently biocompatible may be subjected to surface modifications in order to render the materials biocompatible. Examples of surface modifications include graft polymerization of biocompatible polymers from the material surface, coating of the surface with a crosslinked biocompatible polymer, chemical modification with biocompatible functional groups, and immobilization of a compatibilizing agent such as heparin or other substances. The graft material may also include extracellular matrix materials.

As used herein, a “catheter” is an apparatus that is connected to a deployment mechanism and houses a medical device that can be delivered over a guidewire. The catheter may include a guidewire lumen for over-the-wire guidance and may be used for delivering a stent graft to a target lumen. A catheter can have braided metal strands within the catheter wall for structural improvements. The structural elements of the catheter tip can be bonded or laser welded to the braided strands of the catheter to improve the performance characteristics of the catheter tip.

As used herein, a “guidewire” is an elongated cable comprised of various biocompatible materials including metals and polymers. Guidewires may be used for selecting target lumens and guiding catheters to target deployment locations. Guidewires are typically defined as wires used independently of other devices that do not come as part of an assembly.

As used herein, “lumen” refers to a passage within an arterial structure such as the pulmonary arteries, or stent grafts or the passage within the tubular housings or catheters through which the guidewire may be disposed.

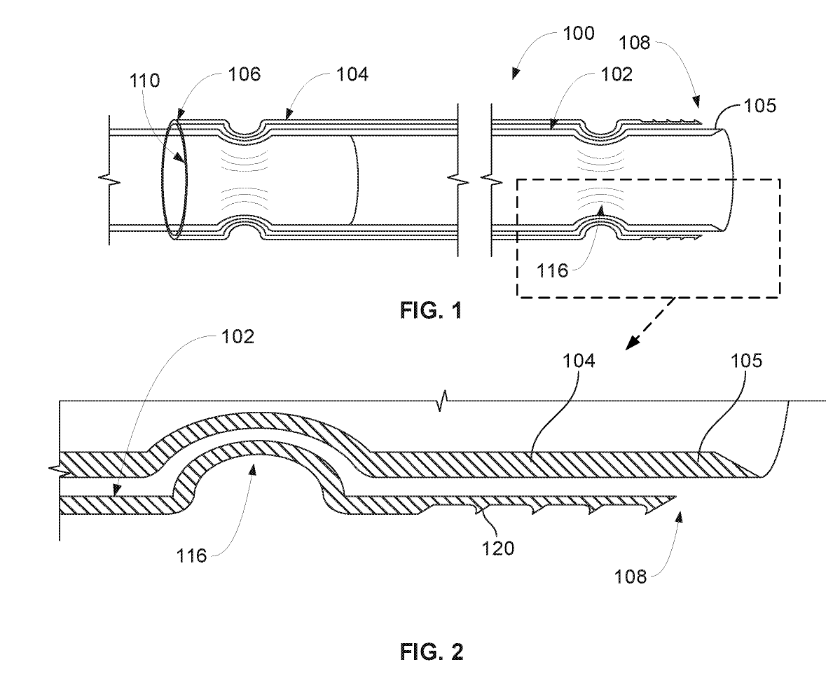

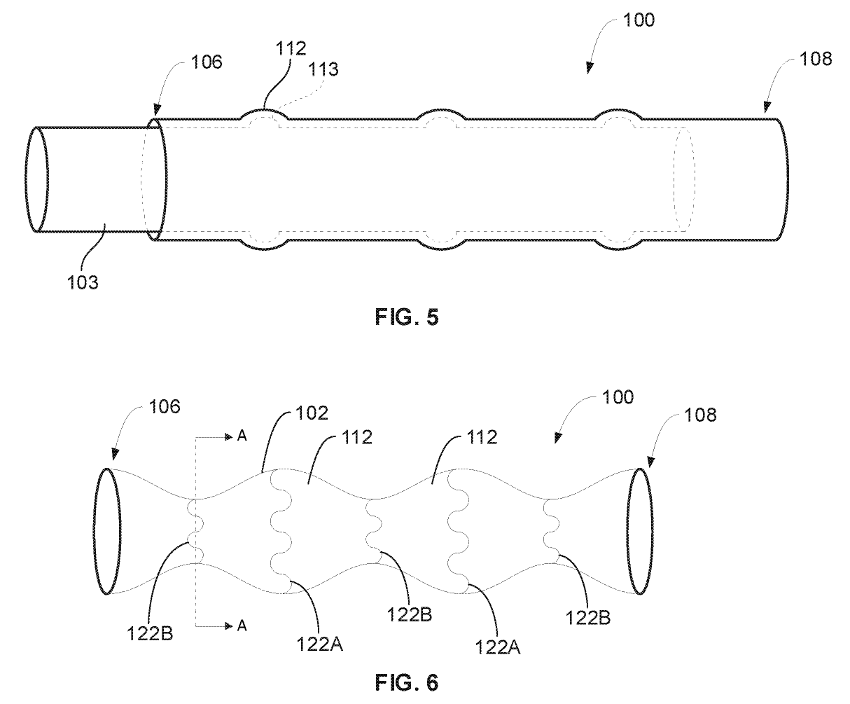

With reference to the Figures, FIG. 1 illustrates a stent graft 100 according to an example embodiment. The stent graft 100 includes a self-expandable stent structure 102 and a graft covering 104 positioned over the self-expandable stent structure 102. The self-expandable stent structure 102 has a first end 106 and a second end 108. The self-expandable stent structure 102 defines a lumen 110. The stent graft 100 further includes at least one annular channel 112 defined by at least one of the self-expandable stent structure 102 and the graft covering 104 and extending radially outward (see FIG. 5) from the self-expandable stent structure 102 or at least one annular protrusion 116 defined by at least one of the self-expandable stent structure 102 and the graft covering 104 and extending radially inward (see FIGS. 1-2, 4) from the self-expandable stent structure 102. In one example, the lumen 110 has a diameter ranging from about 4 mm to about 30 mm and a length ranging from about 20 mm to about 250 mm.

The self-expandable stent structure 102 may comprise a plurality of woven nitinol wires. In such an example, the self-expandable stent structure 102 may further comprise textile fibers intermixed within the woven nitinol wires. In particular, textile fibers can be woven into the nitinol weave in opposing winds. The mix of the two can be optimized in such a way as to match the stretch and compliance of the artery it is supposed to replace. In addition, the outer surface of the self-expandable stent structure 102 can be woven in such a way as to create a wear surface and discourage tissue ingrowth. The inner surface of the self-expandable stent structure 102 can be woven in such a way as to encourage tissue ingrowth to create the process of endothelization. If the fibers on the inner surface of the self-expandable stent structure 102 may be woven in such a way as to align with the direction of blood flow it can further encourage endothelialization. For example, the woven textile filaments may expand when exposed to blood or when exposed to a second component for a binary polymer (e.g., growing a polymer on the stent structure), thereby filling in any gaps within the stent structure. In another example, the self-expandable stent structure 102 further may further comprise a polymer material intermixed within the woven nitinol wires. In yet another example, the self-expandable stent structure 102 comprises a plurality of layers of woven nitinol wires.

Further, as shown in FIG. 2, the graft covering 104 of the main body stent graft may extend about 2 mm to about 3 mm past the self-expandable stent structure 104 at the second end 108 where blood flow exits the lumen 110 after deployment of the stent graft 100 in vivo. The extra length 105 of graft covering 104 may aid with compliance transition from the stent graft 100 to the native vessel. This is beneficial, because if the stent graft 100 has a compliance equal to or less than the branch artery, the result may be the formation of intimal hyperplasia. On the other hand, if the stent graft 100 is more compliant than the native branch vessel, any intimal hyperplasia formation will be reduced. Intimal hyperplasia may eventually lead to stenosis or occlusion of the branch vessel just distal to the bridging stent graft. The blood pressure may be enough to keep the graft covering pressed against the branch vessel wall and maintain seal of the blood in vessels which always have positive velocity. However, some branch arteries may have flow that reverses at certain points in the cardiac cycle. In this instance, stent barbs for active fixation on the distal end of the graft covering 104 may aid in maintaining the blood seal.

In one example, as shown in FIG. 2, the second end 108 of the self-expandable stent structure 102 includes a plurality of extensions 120 biased radially outward. Such extensions 120 may be at least about 5 mm in length, in one example. In one particular embodiment, the plurality of extensions 120 are a plurality of barbs. Such a plurality of barbs may extend from the self-expandable stent structure 102 into the lumen 110 towards the first end 106 of the self-expandable stent structure 102.

The extensions 120 may be similar to cantilevers (as opposed to a circumferential radial effect) and may be biased outward to hold the graft covering 104 in better apposition to the native vessel and for any barbs to resist stent graft pull out. In another embodiment, a plurality of extensions 120 may be used to aid in the cloth-to-vessel apposition.

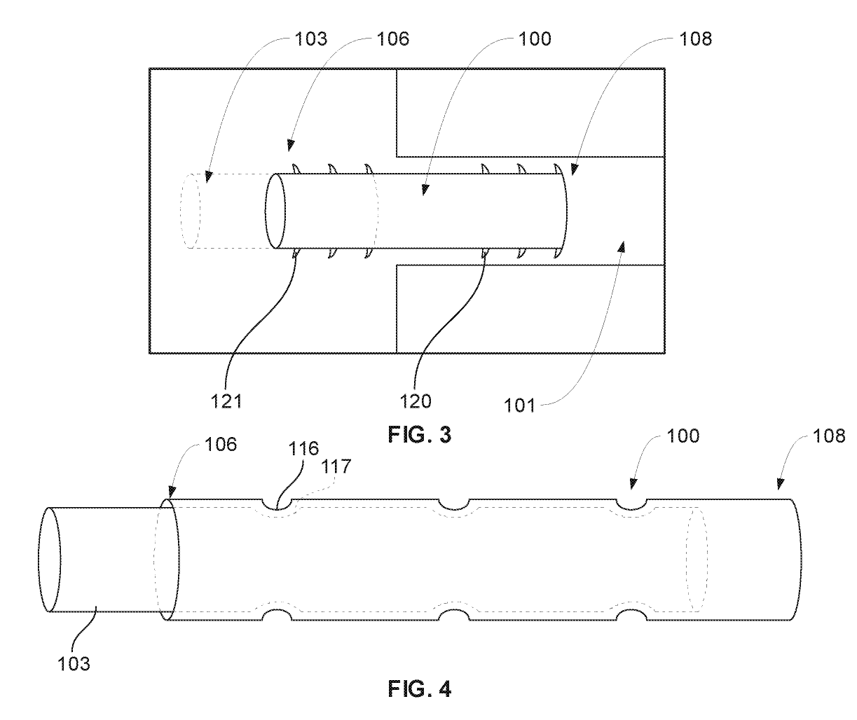

In a further embodiment, the plurality of extensions 120 may have a barbed outer surface, as shown in FIG. 2, to create both a positive fixation between the stent graft 100 and the native artery as well as with any additional bridging stent (thus helping to prevent stent separation). The distal end 108 fixation is important as it helps keep the stent graft 100 from pulling out of the branch artery and the blood from being pumped into the aneurysm sac. Blood flow into the aneurysmal sac could be catastrophic resulting in aneurysm rupture and requiring open surgical intervention to repair. To ensure the second end 108 of the stent graft 100 remains in the branch artery, active fixation is preferred. Extensions 120 that are biased toward the ostium of the branch vessel can traumatically dig into the tissue thereby anchoring the distal end 108 in place. FIG. 3 illustrates a stent graft 100 deployed in a branch artery 101 with extensions 120 on the second end 108 to hold the stent graft 100 in place in the branch artery.

The first end 106 of the stent graft 100 can also benefit from active fixation. In example embodiments for which the first end 106 will be placed in a previously-deployed stent graft 103, opposing stent barbs 121 could be used to create active fixation. Active fixation helps prevent the stent graft 100 from being pulled out of the previously-deployed stent graft 103 and may also allow for shorter stent structures to be employed on the stent graft 100 allowing for less aorta to be covered effectively minimizing the risk of paraplegia.

In another embodiment, the stent graft 100 may further include a plurality of annular channels 112 (FIG. 5) or a plurality of annular protrusions 116 (FIG. 4) arranged as corrugations disposed along at least a portion of the self-expandable stent structure 102. The annular channels 112 or protrusions 116 (i.e., female/male interlock feature) that may permit interlocking with a subsequent bridging stent graft 103. In one example, the at least one annular channel 112 or the at least one annular protrusion 116 is located about 10 mm to about 30 mm from the second end 108 of the self-expandable stent structure 104. In another embodiment, the at least one annular channel 112 or the at least one annular protrusion 116 is located about 10 mm to about 30 mm from the first end 106 of the self-expandable stent structure 104, as shown in FIGS. 4 and 5.

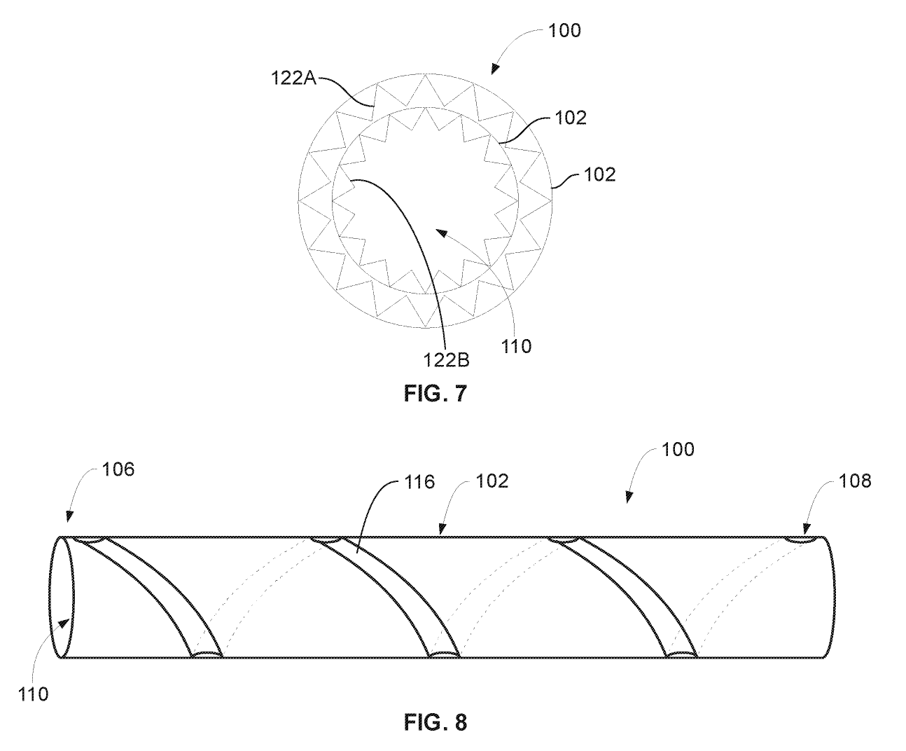

In another embodiment shown in FIGS. 6-7, the stent graft 100 may further include a plurality of annular channels 112 that are result from a plurality of radially directed sinusoidal stents 122A, 122B arranged along at least a portion of the self-expandable stent structure 102. The radially directed sinusoidal stents may comprise a shape memory material, such as nitinol as an example. In one example, every second radially directed sinusoidal stent of the plurality of sinusoidal stents has a stronger radial force than that of adjacent sinusoidal stents. As shown in FIGS. 6 and 7, the radially directed sinusoidal stents 122A have a stronger radial force than the radially directed sinusoidal stents 122B. In one example, every second radially directed sinusoidal stent of the plurality of sinusoidal stents has a different diameter than that of adjacent sinusoidal stents. For example, as shown in FIGS. 6 and 7, the radially directed sinusoidal stents 122A have a larger diameter than the radially directed sinusoidal stents 122B. Such a configuration may provide zones of alternating outward radial force along the length of the stent graft 100. The alternating zones of outward radial force can form interlocking zones between overlapping bridging stents. If the zones of alternating outward radial force create annular channels 112, it will not cause long term flow disturbances, because the annular channel 112 will fill with thrombus over time. In another example, every second radially directed sinusoidal stent of the plurality of sinusoidal stents has a radial opacity different from that of adjacent sinusoidal stents. Interlocking features may have variable lengths of overlap to achieve a desired overall length.

Further, the at least one annular channel 112 or the at least one annular protrusion 116 in the surface of the lumen 110 may be helical in shape. FIG. 8 illustrates a helical protrusion 116 in the inner surface 114 of the lumen 110. Helical channels and/or protrusions 112, 116 may helpful for two reasons. First, helical channels and/or protrusions may aid with redeveloping turbulent bloodflow before the flow reaches the uncovered portions of the branch vessel. This can be important because when the blood flow departs the aortic channel for the branch vessel it becomes disturbed. If the flow remains disturbed as it passes through the stent graft 100 and once it reaches the uncovered branch vessel, the disturbed blood flow can cause intimal hyperplasia. If helical channels and/or protrusions 112, 116 are involved it can cause the blood to travel a helical path which is effectively longer than a straight axial path, giving the blood a longer distance along which to develop, ultimately improving the odds of long term branch vessel patency. Second, these helical channels and/or protrusions 112, 116 may act as a male/female locking mechanism and increase contact surface area of two stent grafts to achieve greater passive and active fixation, ultimately preventing stent graft separation.

In one embodiment, a pair of opposing helical stent structures may be coupled to and extend along the length of lumen 110 of the self-expandable stent structure 102. The helical stent structures may advantageously prevent elongation of the lumen 110. These helical stent structures may be made from biocompatible materials with elastic shape memory, such as nitinol, stainless steel, plastics, polymers or any combination of such materials, among other possibilities.

In operation, an example method for placement of a stent graft 100 may include (a) introducing a guidewire into an arterial configuration via arterial access, (b) loading a delivery catheter containing the stent graft 100 according to the embodiments described above onto the guidewire, (c) moving the delivery catheter along the guidewire and introducing the delivery catheter into the arterial configuration via arterial access, and (d) deploying the stent graft 100 into at least one of the arterial configuration and a lumen of a previously-placed stent graft. In one embodiment, the method may further include (e) prior to deploying the stent graft 100, aligning the at least one annular channel 112 or the at least one protrusion 116 with a corresponding annular channel 113 or annular protrusion 117 in the previously-placed stent graft via radiopaque markers.

It will be appreciated that other arrangements are possible as well, including some arrangements that involve more or fewer steps than those described above, or steps in a different order than those described above.

While various aspects and embodiments have been disclosed herein, other aspects and embodiments will be apparent to those skilled in the art. All embodiments within and between different aspects of the invention can be combined unless the context clearly dictates otherwise. The various aspects and embodiments disclosed herein are for purposes of illustration and are not intended to be limiting, with the true scope and spirit being indicated by the claims.

1. A stent graft, comprising:
a self-expandable stent structure and a graft covering positioned over the self-expandable stent structure, the self-expandable stent structure having a first end and a second end, wherein the self-expandable stent structure defines a lumen; and
at least one annular channel defined by at least one of the self-expandable stent structure and the graft covering and extending radially outward from the self-expandable stent structure or at least one annular protrusion defined by at least one of the self-expandable stent structure and the graft covering and extending radially inward from the self-expandable stent structure.

2. The stent graft of claim 1, wherein the lumen has a diameter ranging from about 4 mm to about 30 mm and a length ranging from about 20 mm to about 250 mm.

3. The stent graft of claim 1, wherein the graft covering extends about 2 mm to about 3 mm past the second end of the self-expandable stent structure.

4. The stent graft of claim 1, wherein the second end of the self-expandable stent structure comprises a plurality of extensions biased radially outward.

5. The stent graft of claim 4, wherein the plurality of extensions are at least about 5 mm in length.

6. The stent graft of claim 4, wherein the plurality of extensions comprise a plurality of barbs.

7. The stent graft of claim 6, wherein the plurality of barbs extend from the self-expandable stent structure into the lumen towards the first end of the self-expandable stent structure.

8. The stent graft of claim 1, wherein the at least one annular channel or the at least one annular protrusion is located about 10 mm to about 30 mm from the second end of the self-expandable stent structure.

9. The stent graft of claim 1, wherein the at least one annular channel or the at least one annular protrusion is located about 10 mm to about 30 mm from the first end of the self-expandable stent structure.

10. The stent graft of claim 1, wherein the at least one annular channel or the at least one annular protrusion comprise a plurality of annular channels or a plurality of annular protrusions.

11. The stent graft of claim 10, wherein the plurality of annular channels or the plurality of annular protrusions are arranged as corrugations disposed along at least a portion of the self-expandable stent structure.

12. The stent graft of claim 10, wherein the plurality of annular channels are defined by a plurality of radially directed sinusoidal stents arranged along at least a portion of the self-expandable stent structure.

13. The stent graft of claim 12, wherein every second radially directed sinusoidal stent of the plurality of sinusoidal stents has a stronger radial force than that of adjacent sinusoidal stents.

14. The stent graft of claim 12, wherein every second radially directed sinusoidal stent of the plurality of sinusoidal stents has a different diameter than that of adjacent sinusoidal stents.

15. The stent graft of claim 12, wherein every second radially directed sinusoidal stent of the plurality of sinusoidal stents has a radial opacity different from that of adjacent sinusoidal stents.

16. The stent graft of claim 1, wherein the at least one annular channel or the at least one annular protrusion in the surface of the lumen is a helical shape.

17. A method for placement of a stent graft, the method comprising:
introducing a guidewire into an arterial configuration via arterial access;
loading a delivery catheter containing the stent graft according to claim 1 onto the guidewire;
moving the delivery catheter along the guidewire and introducing the delivery catheter into the arterial configuration via arterial access; and
deploying the stent graft into the arterial configuration and/or a lumen of a previously-placed stent graft.

18. The method of claim 17, further comprising:
prior to deploying the stent graft, aligning the at least one annular channel or the at least one protrusion with a corresponding annular channel or annular protrusion in the previously-placed stent graft via radiopaque markers.
